(19) United States
(12) Patent Application Publication
(10) Pub. No.: US 2025/0241873 A1
(43) Pub. Date: Jul. 31, 2025

(54) KETAMINE NASAL SPRAY FORMULATION AND METHODS OF USE

(71) Applicant: INESKET LLC, Lake Forest, IL (US)

(72) Inventor: Danny Loyd TUCK, Chandler, AZ (US)

(73) Assignee: INESKET LLC, Lake Forest, IL (US)

(21) Appl. No.: 19/037,980

(22) Filed: Jan. 27, 2025

Related U.S. Application Data
(60) Provisional application No. 63/552,847, filed on Feb. 13, 2024.
(60) Provisional application No. 63/625,532, filed on Jan. 26, 2024.

(51) Int. Cl. A61K 31/135; A61K 47/10; A61K 47/18; A61K 47/26

(52) U.S. Cl. CPC A61K 31/135; A61K 47/10; A61K 47/183; A61K 47/186; A61K 47/26

(57) ABSTRACT

The invention relates to pharmaceutical compositions comprising ketamine or pharmaceutically acceptable salt thereof for nasal administration, and methods for using such compositions.

### RELATED APPLICATIONS

This application claims priority under 35 U.S.C. § 119(e) to U.S. Provisional Patent Application No. 63/625,532 filed Jan. 26, 2024 and U.S. Provisional Patent Application No. 63/552,847, filed Feb. 13, 2024, each of which is incorporated herein by reference in their entirety.

### BACKGROUND OF THE INVENTION

There is an unmet need for rapid pain relief for acute painful injuries in pre-hospital environments, the emergency department and other monitored settings. Lack of early and adequate pain relief can lead to chronic pain and long-term depression and diminished quality of life. Current treatment options for moderate to severe pain include opioid medications which carry the risks of life-threatening and/or fatal respiratory depression, accidental ingestion, overdose, addiction, abuse and misuse, and risk of medication errors.

For patients with serious and chronic psychiatric conditions such as post-traumatic stress disorder, anxiety and depression and suicidal ideation or behavior, symptoms are often difficult to treat with currently approved treatments and can lead to acute exacerbations, requiring increased monitoring or hospitalization.

More efficacious and more rapid-acting interventions are needed to treat these high-risk populations and help improve long-term outcomes. The present invention satisfies these needs.

### SUMMARY OF THE INVENTION

The disclosure provides for certain hydro-alcoholic composition comprising ketamine, esketamine (S-enantiomer of ketamine), arketamine (R-enantiomer of ketamine), or a pharmaceutically acceptable salt thereof, wherein the composition comprises about 5 wt % to about 25 wt % ketamine or pharmaceutically acceptable salt thereof; a permeation enhancing agent comprising an alkyl glycoside, wherein the composition comprises about 0.1 wt % to about 20 wt % alkyl glycoside; a cosolvent comprising an alkyl glycol, wherein composition comprises about 1 wt % to about 20 wt % alkyl glycol; an alcohol, wherein composition comprises about 1 wt % to about 20 wt % alcohol; optionally, a preservative agent; optionally, a stabilizing agent; optionally, a buffering agent; and a pharmaceutically acceptable excipient, wherein the excipient comprises water, and the composition comprises about 40 wt % to about 90 wt % water; wherein the hydro-alcoholic composition has a pH of about 3 to about 6.5.

In some embodiments, a hydro-alcoholic may comprise ketamine or a pharmaceutically acceptable salt thereof, wherein the composition comprises about 5 wt % to about 25 wt % ketamine hydrochloride; a permeation enhancing agent comprising about 0.1 wt % to about 20 wt % n-dodecyl-β-D-maltoside; a cosolvent comprising about 1 wt % to about 20 wt % propylene glycol; ethanol comprising about 1 wt % to about 20 wt %; optionally, a preservative agent; optionally, a stabilizing agent; optionally, a buffering agent or pH modifier; and a pharmaceutically acceptable excipient, wherein the excipient comprises water, and the composition comprises about 40 wt % to about 90 wt % water; wherein the hydro-alcoholic composition has a pH of about 3 to about 6.5.

In some embodiments, a hydro-alcoholic may comprise ketamine or a pharmaceutically acceptable salt thereof, wherein the composition comprises about 15 wt % to about 20 wt % ketamine hydrochloride; a permeation enhancing agent comprising about 0.1 wt % to about 1 wt % n-dodecyl-β-D-maltoside; a cosolvent comprising about 1 wt % to about 7 wt % propylene glycol; ethanol comprising about 1 wt % to about 7 wt %; optionally, a preservative agent; optionally, a stabilizing agent; optionally, a buffering agent or pH modifier; and a pharmaceutically acceptable excipient, wherein the excipient comprises water, and the composition comprises about 40 wt % to about 90 wt % water; wherein the hydro-alcoholic composition has a pH of about 3 to about 6.5.

In some embodiments, a hydro-alcoholic may comprise ketamine or a pharmaceutically acceptable salt thereof, wherein the composition comprises about 17 wt % to about 20 wt % ketamine hydrochloride; a permeation enhancing agent comprising about 0.25 wt % to about 1 wt % n-dodecyl-β-D-maltoside; a cosolvent comprising about 4 wt % to about 6 wt % propylene glycol; ethanol comprising about 4 wt % to about 6 wt %; optionally, a preservative agent; optionally, a stabilizing agent; optionally, a buffering agent or pH modifier; and a pharmaceutically acceptable excipient, wherein the excipient comprises water, and the composition comprises about 40 wt % to about 90 wt % water; wherein the hydro-alcoholic composition has a pH of about 3 to about 6.5.

In some embodiments, the hydro-alcoholic composition consists essentially of: about 15 wt % to about 18 wt % ketamine or a pharmaceutically acceptable salt thereof, about 0.3 wt % to about 0.7 wt % n-dodecyl-β-D-maltoside; about 3 wt % to about 7 wt % propylene glycol; about 3 wt % to about 7 wt % ethyl alcohol; about 0.01 wt % to about 0.05 wt % benzalkonium chloride; about 0.01% to about 0.1% ethylenediamine tetraacetate; and water, wherein the composition has a pH of about 3 to about 5.

In some embodiments, the hydro-alcoholic composition consists essentially of about 17 wt % ketamine or a pharmaceutically acceptable salt thereof, about 0.5 wt % n-dodecyl-β-D-maltoside; about 5 wt % propylene glycol; about 5 wt % ethyl alcohol; about 0.02 wt % benzalkonium chloride; about 0.05% ethylenediamine tetraacetate; and water, wherein the composition has a pH of about 3 to about 5.

In some embodiments, the hydro-alcoholic composition consisting of: about 15 wt % to about 20 wt % ketamine or a pharmaceutically acceptable salt thereof; about 0.1 wt % to about 1.5 wt % n-dodecyl-β-D-maltoside; about 1 wt % to about 10 wt % propylene glycol; about 1 wt % to about 10 wt % ethyl alcohol; about 0.01 wt % to about 0.05 wt % benzalkonium chloride; about 0.01% to about 0.1% ethylenediamine tetraacetate; and water, wherein the composition has a pH of about 4 to about 6.

Also provided are methods of using a hydro-alcoholic composition for the treatment of pain, psychiatric disorders, neurological disorders in a subject in need thereof comprising administering to the subject an effective amount of a hydro-alcoholic composition as described herein. In some embodiments, the ketamine pharmaceutically acceptable salt thereof is formulated as a hydro-alcoholic composition as described herein that is suitable for nasal administration.

In some embodiments, a hydro-alcoholic composition is used to treat psychiatric disorders or conditions comprising one or more of generalized anxiety disorder, acute stress disorder, social anxiety disorder, suicidal ideation, acute post-traumatic stress disorder (PTSD) crisis with or without suicidal ideation, PTSD, acute stress disorder/PTSD prevention, acute anxiety disorder, acute and chronic anxiety associated with PTSD, acute agitation, panic disorder, social anxiety disorder, cancer pain induced depression, opioid withdrawal symptoms in patients undergoing buprenorphine initiation/ketamine-assisted buprenorphine initiation, nicotine addiction, alcohol use disorder, addiction disorders, treatment-resistant obsessive-compulsive symptoms in patients unresponsive to first and second line therapies, treatment-resistant anxiety spectrum disorders, depressive episodes in treatment-resistant bipolar depression, treatment-resistant bipolar depression, cocaine use disorder, postpartum depression in women, and postpartum depression in women undergoing cesarean delivery.

In some embodiments, a hydro-alcoholic composition is used to treat pain management and/or as an analgesic to treat one or more of acute pain, neuropathic pain, postoperative pain, complex regional pain syndrome, fibromyalgia, chronic cancer pain, opioid-refractory cancer pain, breakthrough cancer pain, refractory chronic non-cancer pain, opioid-induced hyperalgesia, perioperative pain, and procedural sedation and analgesia for short painful procedures.

In some embodiments, a hydro-alcoholic composition is used to treat a neurological disease or condition comprising one or more of benzodiazepine refractory convulsive status epilepticus, super-refractory status epilepticus, depressive symptoms and cognitive impairment associated with Alzheimer's disease, migraine, migraine in patients who are diagnosed with major depressive disorder, chronic migraine in patients unresponsive to standard preventive and acute migraine therapies, dyskinesia in Parkinson's disease, post-traumatic insomnia, and sleep disturbances in psychiatric disorders.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Illustrates certain permeation characteristics of various embodiments of a hydro-alcoholic composition as described herein.

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a component” includes a plurality of such components, so that a component X includes a plurality of components X. It is further noted that the claims may be drafted to exclude an optional element. As such, this statement is intended to serve as antecedent basis for the use of exclusive terminology, such as “solely,” “only,” “other than”, and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, element, the composition, or the embodiment. The term about can also modify the endpoints of a recited range as discussed above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, an invention encompasses not only the entire group listed as a whole, but each member of the group individually and all possible subgroups of the main group. Additionally, for all purposes, an invention encompasses not only the main group, but also the main group absent one or more of the group members. An invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms.

In various embodiments, the disclosure illustratively described herein may be practiced in the absence of any element, elements, limitation, or limitations, that are not specifically disclosed herein.

As used herein, “subject” refers to a person, an individual, or animal that is the object of medical or scientific study or a patient. In another aspect, the present disclosure provides a composition of matter and method of administrating said composition of matter to a subject, preferably a human, or in a format that can be diluted or reconstituted for administration to the subject.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

For a therapeutic application, an “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is within the capacity of persons skilled in the art. The term “effective amount” is intended to include an amount of a composition described herein, or an amount of a combination of peptides described herein, e.g., that is effective to treat or prevent a disease or disorder, or to treat the symptoms of the disease or disorder, in a host. Thus, an “effective amount” generally means an amount that provides the desired effect.

The term “hydro-alcoholic” refers to a mixture of alcohol and water.

The term “permeation enhancers”, “permeation enhancing agent”, or “absorption enhancer,” as used herein, refers to a functional excipient included in formulations to improve the absorption of a pharmacologically active drug. This term usually refers to an agent whose function is to increase absorption by enhancing nasal mucous-membrane permeation, rather than increasing solubility. In particular, permeation enhancers described herein may improve paracellular transport (i.e., passage through intercellular spaces and tight junctions), transcellular transport (i.e., passive diffusion or active transport across cellular membranes), or transcytosis (i.e., cellular vesicular uptake). Ozsoy et al., Molecules 14:3754-79, 2009.

The term “antimicrobial preservative,” as used herein, refers to a pharmaceutically acceptable excipient with antimicrobial properties which is added to a pharmaceutical composition to maintain microbiological stability.

The term “intranasal administration” in all its grammatical forms refers to administration of a drug through the nasal mucous membrane and/or through the nose-brain pathway directly into the cerebrospinal fluid.

The expression of weight percentage is to be interpreted as % wt./wt. (“wt %”) in this disclosure.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (±)-camphoric acid, camphorsulfonic acid, (±)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (±)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (±)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

A “therapeutic composition” can consist of an admixture with an organic or inorganic carrier or excipient, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary stabilizing, thickening or coloring agents can be used, for example a stabilizing dry agent such as triulose.

As used herein, the term “ketamine” refers to (2-(2-chlorophenyl)-2-(methylamino)-cyclohexanone). Ketamine is a chiral compound. The S(+) and R(−) stereoisomers bind NMDA receptors with different affinities: Kᵢ=3200 and 1100 nM, respectively. As used herein, “ketamine” includes preparations of ketamine that contain a racemic mixture of S(+) and R(−) stereoisomers of ketamine, preparations that contain differences in the enantiomeric proportions of the S(+) and R(−) stereoisomers, and preparations that contain only one (or substantially one) of the enantiomers (e.g., only S(+) ketamine or only R(−) ketamine). S(+)-ketamine may also be referred to as “(S)-ketamine”, “esketamine”, or “Es/ketamine”). R(+)-ketamine may also be referred to as “(R)-ketamine” or “arketamine”.

While in some embodiments, the ketamine and/or esketamine and/or arketamine is described as an HCL salt form, the salt moiety may be interchanged with another salt. In other embodiments, the ketamine, esketamine, and/or arketamine does not include a salt form or is substantially free of a salt form.

### Embodiment of the Invention

The disclosure generally provides for compositions formulated for nasal spray administration comprising ketamine or a pharmaceutically acceptable salt thereof, formulated for quick and effective absorption after administration to a subject requiring, for example, psychiatric uses to include but not limited to treatment of depression, epileptic seizures, post-traumatic stress disorder, suicidal ideation or behavior, addiction, and withdrawal, pain management, anesthesia to include but not limited to, procedural sedation, and acute pain. In particular, the disclosed composition may be formulated for intranasal administration wherein the ketamine or pharmaceutically acceptable salt thereof has greater than 45% absolute bioavailability after administration.

Generally, an exemplary composition is a hydro-alcoholic composition comprising ketamine or a pharmaceutically acceptable salt thereof; a permeation enhancing agent comprising an alkyl glycoside; a cosolvent comprising an alkyl glycol; an alcohol; a preservative agent; a stabilizing agent; a buffering agent; and a pharmaceutically acceptable excipient, wherein the hydro-alcoholic composition has a pH of about 3 to about 6.5, wherein the composition is a non-aqueous solution. For example, in some embodiments, a hydro-alcoholic composition may comprise ketamine or a pharmaceutically acceptable salt thereof, wherein the composition comprises about 5 wt % to about 25 wt % ketamine; a permeation enhancing agent comprising an alkyl glycoside, wherein the composition comprises about 0.1 wt % to about 20 wt % alkyl glycoside; a cosolvent comprising an alkyl glycol, wherein composition comprises about 1 wt % to about 20 wt % alkyl glycol; an alcohol, wherein composition comprises about 1 wt % to about 20 wt % alcohol; optionally, a preservative agent; optionally, a stabilizing agent; optionally, a buffering agent; and a pharmaceutically acceptable excipient, wherein the excipient comprises water, and the composition comprises about 40 wt % to about 90 wt % water; wherein the hydro-alcoholic composition has a pH of about 3 to about 6.5. Preferably, the hydro-alcoholic composition is formulated as a solution comprising the ketamine or pharmaceutically acceptable salt thereof.

Ketamine is a general anesthetic used by anesthesiologists, veterinarians, and researchers. Pharmacologically, ketamine is a noncompetitive NMDA receptor (NMDAR) antagonist. More specifically, ketamine binds to the allosteric site of the NMDA receptor, effectively inhibiting its channel. At high, fully anesthetic level doses, ketamine has also been found to bind to μ-opioid receptors type 2 in cultured human neuroblastoma cells—however, without agonist activity—and to sigma receptors in rats. Ketamine also interacts with muscarinic receptors, descending monoaminergic pain pathways and voltage-gated calcium channels.

The ketamine containing compositions may be formulated to include ketamine or a pharmaceutically acceptable salt thereof of about 1 wt % to about 30 wt % of the composition, or about 5 wt % to about 20 wt % of the composition, or about 5 wt % to about 19 wt % of the composition, or about 5 wt % to about 18 wt % of the composition, or about 5 wt % to about 17 wt % of the composition. In other embodiments, ketamine or a pharmaceutically acceptable salt of ketamine may be present in a composition in an amount of, for example, about 1 wt %, about 2 wt %, about 3 wt %, about 4 wt %, about 5 wt %, about 6 wt %, about 7 wt %, about 8 wt %, about 9 wt %, about 10 wt % about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, or about 30 wt % of the composition. In some embodiments, the ketamine or a pharmaceutically acceptable salt thereof may be present in a composition in an amount of about 15 wt % to about 25 wt %, about 15 wt % to about 20 wt %, or about 15 wt %, about 16 wt %, about 17 wt %, 18 wt %, about 19 wt %, or about 20 wt %. In some embodiments, ketamine or a pharmaceutically acceptable salt thereof may be present in a composition in an amount of, for example, about 1 wt %, about 2 wt %, about 3 wt %, about 4 wt %, about 5 wt %, about 6 wt %, about 7 wt %, about 8 wt %, about 9 wt %, about 10 wt % about 11 wt %, about 12 wt %, about 13 wt %, about 14 wt %, about 15 wt %, about 16 wt %, about 17 wt %, about 18 wt %, about 19 wt %, or about 20 wt % of the composition. In some embodiments, the ketamine or a pharmaceutically acceptable salt thereof may be present in a composition in an amount of about 15 wt % to about 20 wt %, or about 17 wt %, about 18 wt %, or about 19 wt %.

In some embodiments, the amount of ketamine or a pharmaceutically acceptable salt thereof in a formulation may be about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240, mg/mL, or about 250 mg/mL. In other embodiments, the amount of ketamine or a pharmaceutically acceptable salt thereof in a formulation may be about 10 mg/ml to about 200 mg/ml, about 20 mg/ml to about 50 mg/ml, about 50 mg/ml to about 100 mg/ml, or about 100 mg/mL to about 200 mg/mL

In some embodiments, the ketamine or the pharmaceutically acceptable salt thereof is the S-enantiomer (esketamine). In some embodiments, the ketamine or the pharmaceutically acceptable salt thereof is the R-enantiomer (arketamine).

In some embodiments, a ketamine or pharmaceutically acceptable salt thereof containing composition may include one or more permeation enhancing agents. Preferably, the permeation enhancing agent comprises one or more alky glycosides. As used herein, “alkyl glycoside” refers to any sugar joined by a linkage to any hydrophobic alkyl, as is known in the art. Any “suitable” alkyl glycoside means one that fulfills the limiting characteristics of the invention, i.e., that the alkyl glycoside be nontoxic and nonionic, and that it increases the absorption of a compound when it is administered with the compound via the ocular, nasal, nasolacrimal, inhalation or pulmonary, oral cavity (sublingual or Buccal cell), or CSF delivery route.

Exemplary alkyl glycosides may be synthesized by procedures known in the art, i.e., chemically, as described, e.g., in Rosevear et al., Biochemistry 19:4108-4115 (1980) or Koeltzow and Urfer, J. Am. Oil Chem. Soc., 61:1651-1655 (1984), U.S. Pat. Nos. 3,219,656 and 3,839,318 or enzymatically, as described, e.g., in Li et al., J. Biol. Chem., 266:10723-10726 (1991) or Gopalan et al., J. Biol. Chem. 267:9629-9638 (1992), or purchased commercially.

Exemplary alkyl glycosides may include, but are not limited to: alkyl glycosides, such as octyl, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside (synthesized according to Koeltzow and Urfer, J Am Oil Chem Soc, 61: 1651-1655.); alkyl thiomaltosides, such as heptyl, octyl, dodecyl-, tridecyl-, and tetradecyl-β-D-thiomaltoside (synthesized according to Defaye et al., 247-265 (F. W. Lichtenthaler, ed.) VCH Publishers, New York (1991); Ferenci et al., Bacteriol, 144:7-11 (1980)); alkyl thioglucosides, such as heptyl- or octyl 1-thio α- or β-D-glucopyranoside (Saito et al., Chem. Pharm. Bull. 33:503-508 (1985)); alkyl thiosucroses (Binder et al., Carbohydr. Res. 140:9-20 (1985)); alkyl maltotriosides (synthesized according to Koeltzow and Urfer, J Am Oil Chem Soc, 61: 1651-1655.); long chain aliphatic carbonic acid amides of sucrose β-amino-alkyl ethers; (Austrian Patent 382,381 (1987) to Greber et al.; Chem. Abstr., 108:114719 (1988), and Gruber and Greber, “Reactive Sucrose Derivatives” in Carbohydrates as Organic Raw Materials, (F. W. Lichtenthaler, ed.) VCH Publishers, New York (1991), pp. 95-116 pp. 95-116); derivatives of palatinose and isomaltamine linked by amide linkage to an alkyl chain (Kunz, M., “Sucrose-based Hydrophilic Building Blocks as Intermediates for the Synthesis of Surfactants and Polymers” in Carbohydrates as Organic Raw Materials, (F. W. Lichtenthaler, ed.) VCH Publishers, New York (1991), 127-153); derivatives of isomaltamine linked by urea to an alkyl chain (synthesized according to Kunz); long chain aliphatic carbonic acid ureides of sucrose β-amino-alkyl ethers (synthesized according to Gruber and Greber, pp. 95-116); and long chain aliphatic carbonic acid amides of sucrose β-amino-alkyl ethers (synthesized according to Austrian Patent 382,381 (1987) to Greber et al., Chem. Abstr., 108:114719 (1988) and Gruber and Greber, pp. 95-116). Other permeation enhancers are described, for example, in U.S. Pat. No. 10,265,402 to Maggio et al.; Danielsen et al., Pharmaceutics, 2018, 10, 172-188; Madden et al., Pharmaceutics, 2023, 15, 2119-2134; Maher et al., Advanced Drug Delivery Reviews, 177 (2021), 113925-113974.

In some embodiments, the alkyl glycoside is selected from the group consisting of dodecyl maltoside (n-dodecyl-β-D-maltoside), 6-cyclohexyl-1-hexyl-β-D-maltopyranoside, decyl maltoside (n-decyl-β-D-maltopyranoside), octyl glucoside (n-octyl-β-d-glucoside), decyl glucoside (decyl β-D-glucopyranoside), and lauryl glucoside (dodecyl β-D-glucopyranoside).

The alkyl glycoside may be present in the composition in an amount of about 0.01 wt % to about 20 wt % of the composition, about 0.01 wt % to about 15 wt % of the composition, about 0.01 wt % to about 10 wt % of the composition, about 0.01 wt % to about 5 wt % of the composition, about 0.01 wt % to about 4 wt % of the composition, about 0.01 wt % to about 3 wt % of the composition, about 0.01 wt % to about 2.5 wt % of the composition, about 0.01 wt % to about 2 wt % of the composition, about 0.01 wt % to about 1.5 wt % of the composition, about 0.01 wt % to about 1 wt % of the composition, or about 0.01 wt % to about 0.5 wt % of the composition, or any range therebetween. In some embodiments, the alkyl glycoside is present in the composition in an amount of about 0.1 wt % to about 1.5 wt % of the composition, or about 0.3 wt % to about 0.7 wt %, or about 0.5 wt %. In some preferred embodiments, the alkyl glycoside is n-dodecyl-β-D-maltoside present in an amount of about 0.01 wt % to about 1.5 wt %, about 0.1 wt % to about 1.5 wt % of the composition, or about 0.3 wt % to about 0.7 wt %, or about 0.5 wt %.

In some embodiments, the permeation enhancer comprises one or more of dodecyl maltoside, polysorbate 20, polysorbate 80, sodium lauryl sulfate, cetyl pyridinium chloride, deoxycholic acid, fatty acids, benzyl alcohol, capric acid, ceramides, chitosan, cyclodextrins, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl carnitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, or salt thereof, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol β-D-glucoside, sucrose cocoate, taurocholic acid, taurodeoxycholic acid, taurodihydrofusidic acid, thymol, tricaprylin, triolein, alkylsaccharides, and combination thereof.

In some embodiments, the ratio of ketamine or pharmaceutically acceptable salt thereof to the permeation enhancer is about 0.004 to about 4, about 0.04 to about 0.4, or about 0.029.

In some embodiments, a composition may include a cosolvent comprising one or more alkyl glycols such as, but not limited to, ethylene glycol, propylene glycol, butylene glycol, pentylene glycol, any isomers thereof, or any combinations thereof. Preferably, the alkyl glycol is present in the composition in an amount of about 0.1 wt % to about 20 wt %, about 0.1 wt % to about 15 wt %, about 0.1 wt % to about 10 wt %, about 0.1 wt % to about 8 wt %, about 0.1 wt % to about 5 wt %, or any range therebetween. In some embodiments, the alkyl glycol is present in the composition in an amount of about 1 wt % to about 10 wt % about 1 wt % to about 8 wt %, about 2 wt % to about 5 wt %, or about 5 wt %. In some embodiments, the alkyl glycol is present in the composition in an amount of about 3 wt % to about 6 wt %. In some embodiments, the alkyl glycol is present in the composition in an amount of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %. In some embodiments, the alkyl glycol is selected from the group consisting of ethylene glycol, propylene glycol, butylene glycol, and pentylene glycol. In some embodiments, the ratio of ketamine or pharmaceutically acceptable salt thereof to the alkyl glycol is about 0.04 to 4, about 0.1% to about 1, or about 0.29.

In some embodiments, the alkyl glycol is propylene glycol. In some embodiments, the propylene glycol, is present in the composition in an amount of about 0.1 wt % to about 20 wt %, about 0.1 wt % to about 15 wt %, about 0.1 wt % to about 10 wt %, about 0.1 wt % to about 8 wt %, or about 0.1 wt % to about 5 wt %, or any range therebetween, or the propylene glycol is present in the composition in an amount of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %. In some embodiments, the propylene glycol is present in the composition in an amount of about 3.5 wt % to about 6.5 wt %, or about 5 wt %.

The compositions also may include one or more alcohols. For example, a composition may include one or more of ethyl alcohol, propyl alcohol, butyl alcohol, pentyl alcohol, benzyl alcohol, or any isomers thereof, or any combinations thereof. Preferably, the alcohol is present in the composition in an amount of about 0.1 wt % to about 20 wt %, about 0.5 wt % to about 15 wt %, about 1 wt % to about 10 wt %, or about 1.5 wt % to about 5 wt %. In some embodiments, the alcohol is ethanol (dehydrated, USP). In some embodiments, the ethanol is present in the composition in an amount of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %. In some embodiments, the ethanol is present in the composition in an amount of about 3.5 wt % to about 6.5 wt %, or about 5 wt %. In some embodiments, the ratio of ketamine or pharmaceutically acceptable salt thereof to the alcohol is about 0.04 to 4, about 0.1% to about 1, or about 0.29.

Embodiments of hydro-alcoholic compositions also may include one or more buffering agents. Exemplary buffering agents include, for example, phosphate buffered saline, borate buffered saline, citrate buffer saline, citric acid, sodium chloride, calcium chloride, magnesium chloride, potassium chloride, sodium bicarbonate, zinc chloride, hydrochloric acid, sodium hydroxide, acetic acid, sodium acetate, ammonium hydroxide, ammonium chloride, sodium citrate, carbonic acid, bicarbonate ion, KH₂PO₄, K₂HPO₄, or a combination thereof. In other embodiments, the one or more buffering agents comprise an inorganic buffering system such as a combination of hydrochloric acid and sodium hydroxide, or other inorganic buffer systems that are known in the art.

In some embodiments, the buffering agent comprises about 0.01 wt % to about 5 wt %, or about 0.1 wt % to about 3 wt %, or about 0.1 wt % to about 2 wt %, or about 0.1 wt % to about 1.5 wt %, or about 0.1 wt % to about 1 wt %, or about 0.1 wt % to about 0.75 wt %, or about 0.1 wt % to about 0.5 wt %, or about 0.1 wt % to about 0.25 wt % of the hydro-alcoholic composition. In some embodiments, the buffering agent comprises about 0.1 wt % to about 0.5 wt % of the hydro-alcoholic composition. In other embodiments, the buffering agent comprises about 0.25 wt % of the hydro-alcoholic composition. In some embodiments, the hydro-alcoholic composition comprises about 0.1 wt % to about 0.5 wt % citric acid. In some embodiments, the buffering agent is optional.

Some embodiments also may include an effective amount of a stabilizing agent and/or an antimicrobial preservative. The stabilizing agent or antimicrobial preservative can be, for example, ethylenediamine tetraacetate, disodium edetate, sodium perborate, polyquaternium-1 (e.g., Polyquad® preservative), methyl paraben, propyl paraben, benzoic acid, sodium benzoate, chlorbutanol, benzethonium chloride, benzalkonium (BAK) chloride, sodium chlorite, brimonidine, brimonidine purite, polexitonium, or a combination thereof. Generally, the stabilizing agent and the preservative agent are present in an amount of about 0.01 wt % to about 0.1 wt % and about 0.01 wt % to about 0.2 wt %, respectively, and the amount of stabilizing agent and preservative agent varies by not more than ±5% in a composition.

In some embodiments, the preservative agent comprises benzalkonium chloride and the stabilizing agent comprises ethylenediamine tetraacetate. For example, in some embodiments, the benzalkonium chloride is present in an amount of about 0.01 wt % to about 0.05 wt % of the hydro-alcoholic composition, and the ethylenediamine tetraacetate is present in an amount of about 0.01 wt % to about 0.1 wt % of the hydro-alcoholic composition, and the amount of stabilizing agent and preservative agent varies by not more than ±5% in a composition.

A composition also may comprise one or more pharmaceutically acceptable excipients, carriers, or diluents. Preferably, the pharmaceutically acceptable excipients, carriers, or diluents comprise water. In some embodiments, the composition comprises about 40 wt % to about 90 wt % water, or any range therebetween, and the amount of water varies by not more than ±5% in a composition. In some embodiments, the composition comprises about 66 wt % to about 78 wt % water, or about 66 wt % to about 78 wt % water. In some embodiments, the composition comprises about 60 wt %, about 61 wt %, about 62 wt %, about 63 wt %, about 64 wt %, about 65 wt %, about 66 wt %, about 67 wt %, 68 wt %, about 69 wt %, about 70 wt %, about 71 wt %, about 72 wt %, about 73 wt %, about 74 wt %, about 75 wt %, about 76 wt %, about 77 wt %, or about 78 wt % of water.

Preferably, the composition maintains a pH of about 3.0 to about 7.0, about 3.0 to about 6.0, or about 4.0 to about 7.0, about 4.1 to about 6.9, about 4.2 to about 6.8, about 4.3 to about 6.7, about 4.4 to about 6.6, or about 4.45 to about 6.5. In some embodiments, the pH of the composition is about 4 to about 6.5, or about 3 to about 6.

In some embodiments, the hydro-alcoholic composition includes a viscosity of about 0.2 mPas to about 20.0 mPas, about 0.5 mPas to about 5.0 mPas, or about 2.0 mPas. In some embodiments, the hydro-alcoholic composition includes a density of about 0.6 to about 1.4 g/mL, about 0.9 to about 1.1 g/mL, about 0.95 to 1.08 g/mL, or about 1.04 g/mL.

In some embodiments, a hydro-alcoholic composition comprises ketamine or a pharmaceutically acceptable salt thereof, a permeation enhancing agent comprising an alkyl glycoside; a cosolvent comprising an alkyl glycol; an alcohol; and a pharmaceutically acceptable excipient, wherein the hydro-alcoholic composition has a pH of about 4 to about 6.5.

In some embodiments, a hydro-alcoholic composition consists of ketamine or a pharmaceutically acceptable salt thereof, a permeation enhancing agent comprising an alkyl glycoside; a cosolvent comprising an alkyl glycol; an alcohol; and a pharmaceutically acceptable excipient, wherein the hydro-alcoholic composition has a pH of about 3 to about 6.5.

In some embodiments, a hydro-alcoholic composition comprises ketamine or a pharmaceutically acceptable salt thereof in an amount of about 15 wt % to about 20 wt %; n-dodecyl-β-D-maltoside in an amount of about 0.1 wt % to about 1.5 wt %; propylene glycol in an amount of about 1 wt % to about 10 wt %; ethyl alcohol in an amount of about 1 wt % to about 10 wt %; benzalkonium chloride in an amount of about 0.01 wt % to about 0.05 wt %;ethylenediamine tetraacetate in an amount of about 0.01 wt % to about 0.1 wt %; and water, wherein the hydro-alcoholic composition has a pH of about 3 to about 6.

In some embodiments, a hydro-alcoholic composition comprises ketamine or a pharmaceutically acceptable salt thereof, wherein the composition comprises about 5 wt % to about 25 wt % ketamine or a pharmaceutically acceptable salt thereof, a permeation enhancing agent comprising an alkyl glycoside, wherein the composition comprises about 0.1 wt % to about 2 wt % alkyl glycoside, about 1 wt % to about 20 wt % alky glycol, about 1 wt % to about 20 wt % ethanol; optionally, a preservative agent, a stabilizing agent, and/or a buffering agent, taste-masking agents; viscosity modifiers; antioxidants; a pharmaceutically acceptable excipient comprising water, and wherein the composition comprises about 60 wt % to about 78 wt % water; wherein the hydro-alcoholic composition has a pH of about 3 to about 6.5.

In some embodiments, a hydro-alcoholic composition comprises ketamine or a pharmaceutically acceptable salt thereof, wherein the composition comprises about 15 wt % to about 20 wt % ketamine or a pharmaceutically acceptable salt thereof, a permeation enhancing agent comprising an alkyl glycoside, wherein the composition comprises about 0.2 wt % to about 0.9 wt % alkyl glycoside, about 3 wt % to about 7 wt % alky glycol, about 3 wt % to about 7 wt % ethanol; optionally, a preservative agent, a stabilizing agent, and/or a buffering agent; a pharmaceutically acceptable excipient comprising water, and wherein the composition comprises about 60 wt % to about 78 wt % water; wherein the hydro-alcoholic composition has a pH of about 3 to about 6.5.

In some embodiments, a hydro-alcoholic composition comprises about 17 wt % to about 20 wt % ketamine or a pharmaceutically acceptable salt thereof, about 0.5 wt % to about 1 wt % n-dodecyl-β-D-maltoside; about 5 wt % propylene glycol; about 5 wt % ethyl alcohol; about 0.02 wt % benzalkonium chloride; about 0.05% ethylenediamine tetraacetate; optionally, one or more buffering agents; and about 60 wt % to about 78 wt % water, wherein the composition has a pH of about 3 to about 6.

In some embodiments, a hydro-alcoholic composition comprises about 17 wt % to about 19 wt % ketamine or a pharmaceutically acceptable salt thereof, about 0.75 wt % n-dodecyl-β-D-maltoside; about 5 wt % propylene glycol; about 5 wt % ethyl alcohol; about 0.02 wt % benzalkonium chloride; about 0.05% ethylenediamine tetraacetate; optionally, one or more buffering agents; and about 60 wt % to about 78 wt % water, wherein the composition has a pH of about 3 to about 6.

In some embodiments, a hydro-alcoholic composition comprises about 17 wt % to about 19 wt % ketamine or a pharmaceutically acceptable salt thereof, about 0.75 wt % n-dodecyl-β-D-maltoside; about 5 wt % propylene glycol; about 5 wt % ethyl alcohol; about 0.02 wt % benzalkonium chloride; about 0.05% ethylenediamine tetraacetate; optionally, one or more buffering agents; and about 66 wt % to about 78 wt % water, wherein the composition has a pH of about 3 to about 6.

In some embodiments, a hydro-alcoholic composition consists essentially of ketamine or a pharmaceutically acceptable salt thereof in an amount of about 15 wt % to about 18 wt %; n-dodecyl-β-D-maltoside in an amount of about 0.3 wt % to about 0.7 wt %; propylene glycol in an amount of about 3 wt % to about 7 wt %; ethyl alcohol in an amount of about 3 wt % to about 7 wt %; benzalkonium chloride in an amount of about 0.01 wt % to about 0.05 wt %; ethylenediamine tetraacetate in an amount of about 0.01% to about 0.05%; and water, wherein the composition has a pH of about 3 to about 5.

In some embodiments, a hydro-alcoholic composition consists essentially of ketamine or a pharmaceutically acceptable salt thereof in an amount of about 17 wt %; n-dodecyl-β-D-maltoside in an amount of about 0.5 wt %; propylene glycol in an amount of about 5 wt %; ethyl alcohol in an amount of about 5 wt %; benzalkonium chloride in an amount of about 0.02 wt %; ethylenediamine tetraacetate in an amount of about 0.05 wt %; and water, wherein the composition has a pH of about 3 to about 5.

In some embodiments, a hydro-alcoholic composition consists of ketamine or a pharmaceutically acceptable salt thereof in an amount of about 15 wt % to about 18 wt %; n-dodecyl-β-D-maltoside in an amount of about 0.3 wt % to about 0.7 wt %; propylene glycol in an amount of about 3 wt % to about 7 wt %; ethyl alcohol in an amount of about 3 wt % to about 7 wt %; benzalkonium chloride in an amount of about 0.01 wt % to about 0.05 wt %; ethylenediamine tetraacetate in an amount of about 0.01% to about 0.05%; and water, wherein the composition has a pH of about 3 to about 5.

In some embodiments, a hydro-alcoholic composition consists of ketamine hydrochloride or a pharmaceutically acceptable salt thereof in an amount of about 17 wt %; n-dodecyl-β-D-maltoside in an amount of about 0.5 wt %; propylene glycol in an amount of about 5 wt %; ethyl alcohol in an amount of about 5 wt %; benzalkonium chloride in an amount of about 0.02 wt %; ethylenediamine tetraacetate in an amount of about 0.05 wt %; and water, wherein the composition has a pH of about 3 to about 5.

In some embodiments, a hydro-alcoholic composition consists of about 18 wt % ketamine or a pharmaceutically acceptable salt thereof; about 0.5 wt % n-dodecyl-β-D-maltoside; about 5 wt % propylene glycol; about 5 wt % ethyl alcohol; about 0.01 wt % benzalkonium chloride; about 0.05% ethylenediamine tetraacetate; optionally, one more buffering agents; and water, wherein the composition has a pH of about 3 to about 5.

In some embodiments, a hydro-alcoholic composition consists of about 19 wt % ketamine or a pharmaceutically acceptable salt thereof; about 0.5 wt % n-dodecyl-β-D-maltoside; about 5 wt % propylene glycol; about 5 wt % ethyl alcohol; about 0.01 wt % benzalkonium chloride; about 0.05% ethylenediamine tetraacetate; optionally, one more buffering agents; and water, wherein the composition has a pH of about 3 to about 5.

The compositions and formulations described herein may be for administration by oral (solid or liquid), parenteral (intramuscular, intraperitoneal, intravenous (IV) or subcutaneous injection), transdermal (either passively or using ionophoresis or electroporation), transmucosal (nasal, intranasal, vaginal, rectal, or sublingual), or inhalation routes of administration, or using bioerodible inserts and can be formulated in dosage forms appropriate for each route of administration. The most suitable route in any given case will depend on the particular host, and nature and severity of the conditions for which the active ingredient is being administered. The compositions may be conveniently presented in either unit or multi dosage form and prepared by any of the methods well known in the art of pharmacy and using well known carriers and excipients.

In some embodiments, the route of administration of ketamine or pharmaceutical acceptable salt thereof is intravenous (IV). Ketamine or pharmaceutical acceptable salt thereof may thus also be prepared in a formulation or pharmaceutical composition appropriate for IV administration. Ketamine or pharmaceutical acceptable salt thereof can be mixed with a pharmaceutically acceptable carrier or excipient. By way of example, ketamine or pharmaceutical acceptable salt thereof can be formulated in a saline solution for intravenous administration.

A preferred mode of administration is intranasal administration, i.e., through the nasal mucosa and/or through the nose-brain pathway directly into the cerebrospinal fluid is described in Wen et al., Discov Med, 2011, 11:497-503, is hereby incorporated by reference in its entirety. As discussed in Wen, drugs administered intranasally may reach the brain via alternatives pathways. In one pathway, drugs, e.g., ketamine or esketamine, are absorbed systemically, following absorption through the blood vessels of the nasal respiratory epithelium. Drugs delivered via this systemic pathway must first cross the blood brain barrier, prior to reaching the brain. In an alternative delivery pathway, drugs administered intranasally can be rapidly transported into the CNS via the connection between the olfactory epithelium at the roof of the nasal cavity and the trigeminal system of the brain. This affords a direct connection, with no synapse between the olfactory neurons and the brain. The pathway thus allows transport of active agents to the brain without passage through the blood brain barrier.

Nasal formulations may be administered with the aid of a delivery device, e.g., an aerosol delivery. Any form of aerosolization known in the art, including but not limited to spray bottles, nebulization, atomization or pump aerosolization of a liquid formulation, and aerosolization of a dry powder formulation, can be used.

Nasal formulations may be administered, for example, using a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation.

A useful device for intranasal administration is a small, hard bottle to which a metered dose sprayer is attached. In one embodiment, the metered dose is delivered by drawing the ketamine solution into a chamber of defined volume, which chamber has an aperture dimensioned to aerosolize and aerosol formulation by forming a spray when a liquid in the chamber is compressed. The chamber is compressed to administer the ketamine. In a specific embodiment, the chamber is a piston arrangement. In some embodiments, the assembled device comprises a reservoir, a plunger, a cannula, a spray pin, a reservoir holder and an actuator. Such devices are commercially available.

Other devices useful for administering a dose intranasally are mucosal automation device that provide atomization of topical solution across the nasal and oropharyngeal mucous membranes that produce a typical particle size of 30 microns. An example of such a device is the LMA MAD Nasal™ device (LMA Company, San Diego, Calif.), which produces a typical particle size of 30 microns, has a system dead space of 0.09 mL, a tip diameter of about 3/16″ (4 mm), and an applicator length of about 1¾″ (44 mm) can be used.

In some embodiments, an apparatus or device may comprise, for example, at least one fluid reservoir and a head portion, wherein the head portion is adapted for nasal administration of a hydro-alcoholic composition as described herein. In some embodiments, the device may be used to limit the amount of formulation delivered per administration by, for example, having a small reservoir that only holds a specific amount of formulation (e.g., about 5 μl, about 10 μl, about 15 μl, about 20 μl, about 25 μl, about 30 μl, about 40 μl, about 50 μl, etc.).

In some embodiments, the assembled device delivers one or more doses of the hydro-alcoholic composition with unit dose device or multidose device. In some embodiments, the assembled device is a unit-dose device that delivers one dose of the hydro-alcoholic composition upon a single actuation and comprises a single reservoir containing not more than 200 μL of the hydro-alcoholic composition or the single reservoir contains about 140 μL to 180 μL of the hydro-alcoholic composition. In some embodiments, the assembled device delivers about 120 μL to 130 μl of the hydro-alcoholic composition upon a single actuation. In some embodiments, the assembled device delivers about 120 μL of the hydro-alcoholic composition upon the single actuation and comprises a single reservoir containing about 150 μL of the hydro-alcoholic composition. In some embodiments, the assembled device delivers about 125 μL of the hydro-alcoholic composition upon the single actuation and comprises a single reservoir containing about 155 μL of the hydro-alcoholic composition. In some embodiments, the assembled device delivers about 130 μL of the hydro-alcoholic composition upon the single actuation and comprises a single reservoir containing about 160 μL of the hydro-alcoholic composition.

In some embodiments, the assembled device is a unit-dose device that delivers one dose of the hydro-alcoholic composition upon a single actuation when the stopper height is set between 15.7 mm and 16.7 mm and comprises a single reservoir containing not more than 200 μL of hydro-alcoholic composition. In some embodiments, the assembled device is a unit-dose device that delivers about 120 μL to 130 μL of the ketamine formulation upon a single actuation when the stopper height is set between 15.7 mm and 16.7 mm and comprises a single reservoir containing not more than 180 μL of the hydro-alcoholic composition.

In some embodiments, the assembled device is a unit-dose device that delivers about 120 μL of the hydro-alcoholic composition upon a single actuation when the stopper height is set about 15.7±0.5 mm and comprises a single reservoir containing about 150±10 μL of the hydro-alcoholic composition. In some embodiments, the assembled device is a unit-dose device that delivers about 125 μL of the hydro-alcoholic composition upon a single actuation when the stopper height is set about 16.2±0.5 mm and comprises a single reservoir containing about 155±10 μL of the hydro-alcoholic composition. In some embodiments, the assembled device is a unit-dose device that delivers about 130 μL of the hydro-alcoholic composition upon a single actuation when the stopper height is set about 16.2±0.5 mm and comprises a single reservoir containing about 160±10 μL of the hydro-alcoholic composition.

In some embodiments, the hydro-alcoholic composition provides a spray pattern characteristic of a Dmin (shortest diameter of the spray pattern) from about 42 to about 66 millimeters, a Dmax (longest diameter of the spray pattern) from about 58 to about 79 millimeters, an ovality ratio from about 0.5 to 3 at about at 5 centimeters from a spray nozzle. Dmin from about 31 to about 50 millimeters, a Dmax from about 43 to about 68 millimeters, an ovality ratio from about 0.5 to 3 about at 3 centimeters from a spray nozzle and a combination thereof.

In some embodiments, the hydro-alcoholic composition provides a particle size parameter at 3 centimeters from a spray nozzle, of a DV (10) from about 10 to about 20 microns, a DV (50) from about 20 to about 60 microns, a DV (90) from about 60 to about 150 microns, and/or a span from about 1 to about 4, or a combination thereof. DV refers to the volume distributions from the droplet size (particle size) experiments. DV10, DV50, and DV90 are droplet size values representing the diameter below which 10%, 50%, and 90% of the total droplet volume exists in a sample, respectively; essentially indicating the distribution of droplet sizes. Methods of determining the aforementioned characteristics are known in the art, and are described, for example in Dayal et al., J Pharm Sci. 2004 July; 93(7):1725-42 and Makidon et al., J Aerosol Med Pulm Drug Deliv. 2010 April; 23(2):77-89.

### Therapeutic Uses.

The disclosure also provides for methods of treating various diseases and/or conditions using the disclosed compositions to deliver therapeutically effective amounts of ketamine or a pharmaceutically acceptable salt thereof to a subject in order to treat said disease and/or condition.

In some embodiments, a hydro-alcoholic composition is used to treat a psychiatric disorder or conditions such as generalized anxiety disorder, acute stress disorder, social anxiety disorder, suicidal ideation, acute post-traumatic stress disorder (PTSD) crisis with or without suicidal ideation, PTSD, acute stress disorder/PTSD prevention, acute anxiety disorder, acute and chronic anxiety associated with PTSD, acute agitation, panic disorder, social anxiety disorder, cancer pain induced depression, opioid withdrawal symptoms in patients undergoing buprenorphine initiation/ketamine-assisted buprenorphine initiation, nicotine addiction, alcohol use disorder, addiction disorders, treatment-resistant obsessive-compulsive symptoms in patients unresponsive to first and second line therapies, treatment-resistant anxiety spectrum disorders, depressive episodes in treatment-resistant bipolar depression, treatment-resistant bipolar depression, cocaine use disorder, postpartum depression in women, and postpartum depression in women undergoing cesarean delivery.

In some embodiments, a hydro-alcoholic composition is used to treat pain or for pain management and/or as an analgesic to treat one or more of acute pain, neuropathic pain, postoperative pain, complex regional pain syndrome, fibromyalgia, chronic cancer pain, opioid-refractory cancer pain, breakthrough cancer pain, refractory chronic non-cancer pain, opioid-induced hyperalgesia, perioperative pain, and procedural sedation and analgesia for short procedures.

In some embodiments, a hydro-alcoholic composition is used to treat a neurological disease or condition such as benzodiazepine refractory convulsive status epilepticus, super-refractory status epilepticus, depressive symptoms and cognitive impairment associated with Alzheimer's disease, migraine, migraine in patients diagnosed with major depressive disorder, chronic migraine in patients unresponsive to standard preventive and acute migraine therapies, dyskinesia in Parkinson's disease, post-traumatic insomnia, and sleep disturbances in psychiatric disorders.

In some embodiments, a hydro-alcoholic composition is used as adjuvant therapy for cancers to inhibit tumor growth and progression.

### Therapeutic Dosages.

Preferably, for use of the hydro-alcoholic composition to treat the various diseases and/or conditions, the hydro-alcoholic composition is formulated as a nasal spray formulation to provide up to 50 milligrams of ketamine or a pharmaceutically acceptable salt thereof per spray, up to 45 milligrams of ketamine or a pharmaceutically acceptable salt thereof per spray, up to 40 milligrams of ketamine or a pharmaceutically acceptable salt thereof per spray, up to 35 milligrams of ketamine or a pharmaceutically acceptable salt thereof per spray, up to 30 milligrams of ketamine or a pharmaceutically acceptable salt thereof per spray, up to 25 milligrams of ketamine or a pharmaceutically acceptable salt thereof per spray, up to 20 milligrams of ketamine or a pharmaceutically acceptable salt thereof per spray, up to 15 milligrams of ketamine or a pharmaceutically acceptable salt thereof per spray, up to 10 milligrams of ketamine or a pharmaceutically acceptable salt thereof per spray, or up to 5 milligrams of ketamine or a pharmaceutically acceptable salt thereof per spray. In some embodiments, the effective amount of composition comprising only the S-enantiomer of the R-enantiomer is half the amount of ketamine or pharmaceutically acceptable salt thereof. For example, if the effective amount of ketamine administered to a subject is about 20 mg or 40 mg per administration, then the effective amount of S-ketamine or R-ketamine is 10 mg and 20 mg, respectively.

In some embodiments, about 0.1 mg to about 1000 mg of ketamine or a pharmaceutically acceptable salt thereof, may be administered to a subject, as a hydro-alcoholic composition, over about a 24-hour period, over about a 22-hour period, over about a 20-hour period, over about a 18 hour period, over about a 16 hour period, over about a 14-hour period, over about a 12 hour period, over about a 10 hour period, over about an 8-hour period, over about a 6 hour period, over about a 4 hour period, over about a 2 hour period, over about a 1-hour period.

In some embodiments, the composition may be administered every hour, 2 hours, 3 hours, 4, hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, every day, every 2 days, every 3 days, every 4 days, every 7 days, every 10 days, every 14 days or every 30 days. In some embodiments, the composition is administered at least 1, 2, 3, 4, 5, 6, 7, 8, 9, or 10 times per week for a duration of about 1 week, about 2 weeks, about 3 weeks, about 4 weeks, about 5 weeks, about 6 weeks, about 7 weeks, about 8 weeks, about 9 weeks, about 10 weeks, about 15 weeks, about 20 weeks, about 25 weeks, about 30 weeks, about 35 weeks, about 40 weeks, about 45 weeks, about 50 weeks, about 52 weeks, or greater than 52 weeks. In some embodiments, the hydro-alcoholic composition is administered about 3 times per week for about 12 weeks or for about 52 weeks.

In some embodiments, the absolute bioavailability of the ketamine or a pharmaceutically acceptable salt thereof is 40% or greater, 45% or greater, 50% or greater, 55% or greater, 60% or greater, 65% or greater, 70% or greater, 75% or greater, 80% or greater, 85% or greater, 90% or greater, or 95% or greater. Methods of determining absolute bioavailability are known in the art. For example, to determine absolute bioavailability, the tested drug and mode of administration is measured against an intravenous reference dose. The bioavailability of the intravenous dose is 100% by definition. For example, animals or volunteering humans are given an intravenous injection and corresponding oral doses of a drug. Urinary or plasma samples are taken over a period of time and levels of the drug over that period of time are determined. (See DeMuro et al., (2000) J Clin. Pharmacol. 40:781-784.)

In some embodiments, the ketamine or a pharmaceutically acceptable salt thereof is administered intranasally, as a hydro-alcoholic composition, in an amount of about 5 mg per spray to about 30 mg per spray, about 10 mg per spray to about 25 mg per spray, about 17 mg per spray to about 22 mg per spray, or any amount or range therein.

In some embodiments, the amount of administered ketamine or a pharmaceutically acceptable salt thereof, delivered as a hydro-alcoholic composition, is about 0.01 mg to about 1500 mg, or any amount or range therein, about 0.01 mg to about 1250 mg, or any amount or range therein, about 0.01 mg to about 1000 mg, or any amount or range therein, about 0.01 mg to about 500 mg, or any amount or range therein, about 0.1 mg to about 250 mg, or any amount or range therein, about 0.01 mg to about 100 mg, or any amount or range therein, about 0.01 mg to about 50 mg, or any amount or range therein, or about 0.01 mg to about 25 mg, or any amount or range therein. In some embodiments, the amount of administered ketamine or a pharmaceutically acceptable salt thereof delivered as a hydro-alcoholic composition, is, about 0.01 mg, about 0.025 mg, about 0.05 mg, about 0.1 mg, about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 95 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg.

In some embodiments, the amount of ketamine or a pharmaceutically acceptable salt thereof that is administered as a hydro-alcoholic composition is about 1 to about 25 mg/kg of body weight, about 1 to about 20 mg/kg of body weight, about 1 to about 15 mg/kg of body weight, about 1 to about 10 mg/kg of body weight, about 1 to about 5 mg/kg of body weight, or any range therebetween. In some embodiments, the amount of ketamine or a pharmaceutically acceptable salt thereof that is administered as a hydro-alcoholic composition is about 0.01 to about 2 mg/kg of body weight, about 0.01 to about 1.5 mg/kg of body weight, about 0.05 to about 1.4 mg/kg of body weight, about 0.05 to about 1.3 mg/kg of body weight, about 0.05 to about 1.2 mg/kg of body weight, approximately 0.05 to about 1.1 mg/kg of body weight, about 0.01 to about 1 mg/kg of body weight, about 0.05 to about 0.7 mg/kg of body weight, or any amount or range therein. Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.) and Nair et al., J Basic Clin Pharma 2016; 7:27-31. The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In some embodiments, a total volume of the spray per administration is about 25 μL to about 500 μL, about 25 μL to about 450 μL, about 25 μL to about 400 μL, about 25 μL to about 350 μL, about 25 μL to about 300 μL, about 25 μL to about 250 μL, about 25 μL to about 200 μL, about 25 μL to about 150 μL, or about 25 μL to about 100 μL. In some embodiments, the total volume of the spray is about 50 μL to about 200 μL, about 50 μL to about 150 μL, or any range between a given range.

In some embodiments, a disease or condition may be treated by intranasally administering to the subject an effective amount of the hydro-alcoholic composition, wherein the hydro-alcoholic composition comprising about 15 wt % to about 20 wt % of ketamine or a pharmaceutically acceptable salt thereof in a first nasal administration to deliver about 10 mg to about 160 mg of ketamine or a pharmaceutical salt thereof in an amount of about 50 μL to about 200 μL. In some embodiments, a second nasal administration is conducted bilaterally. In some embodiments, after a waiting period after the first administration, a second administration of the hydro-alcoholic composition comprising about 15 wt % to about 20 wt % ketamine or a pharmaceutically acceptable salt thereof, may be administered to the same nostril or via a bilateral administration. In some embodiments, the waiting period is about 10 seconds to about 30 minutes, or about 10 seconds to about 60 minutes. In some embodiments, about 10 mg to 200 mg, about 10 mg to about 160 mg, about 15 mg to about 80 mg, or about 20 mg to about 40 mg of ketamine or a pharmaceutically acceptable salt thereof are delivered intranasally to the subject in about 1 to about 8 administrations. In some embodiments, about 20 mg, about 40 mg, about 80 mg, or about 160 mg of ketamine or a pharmaceutically acceptable salt thereof is delivered intranasally to the subject in from about 1 to about 8 administrations. In preferred embodiments, a single nasal administration delivers about 10 mg to about 40 mg of ketamine of a pharmaceutical salt thereof is a volume of about 50 μL to about 200 μL.

In some embodiments, a method a disease or condition in a subject in need thereof comprising intranasally administering to the subject an effective amount of a hydro-alcoholic composition, wherein the effective amount the hydro-alcoholic composition is sufficient to deliver about 1 mg to about 500 mg of ketamine or a pharmaceutically acceptable salt thereof in about 1 to about 10 nasal administrations in about a 15-minute to about a 120-minute period. In some embodiments, the number of nasal administrations is 1, 2, 3, 4, 5, 6, 7, 8, 9, or 10 or more.

In some embodiments, treatment of a psychiatric disorder or condition, such as generalized anxiety disorder, social anxiety disorder, suicidal ideation, acute post-traumatic stress disorder (PTSD) crisis with or without suicidal ideation, PTSD, alcohol use disorder, addiction disorder, nicotine addiction, treatment-resistant obsessive-compulsive symptoms in patients unresponsive to first and second line therapies, treatment-resistant anxiety spectrum disorders, depressive episodes in treatment-resistant bipolar depression, treatment-resistant bipolar depression, cocaine use disorder, postpartum depression in women, postpartum depression in women undergoing cesarean, treatment of cancer pain induced depression, acute stress disorder, acute anxiety, and acute and chronic anxiety associated with PTSD include a treatment protocol of dosing about 1 to about 4 times per week, for about 2 to about 12 weeks, optionally followed by a maintenance period including dosing every 1 to about 8 weeks as needed. In some embodiments, for acute stress disorder/PTSD prevention, comprises dosing immediately upon triage at an emergency facility with 2nd dose 24 hours later, and optionally additional doses as needed.

In some embodiments, pain treatment and/or as an analgesic treatment of neuropathic pain, complex regional pain syndrome, fibromyalgia, chronic cancer pain, opioid-refractory cancer pain, breakthrough cancer pain, refractory chronic non-cancer pain, and opioid-induced hyperalgesia include a treatment protocol of dosing about 1 to about 4 times per week for about 2 to about 12 weeks, optionally followed by a maintenance period including dosing every 1 to about 8 weeks as needed, at 1 to about 6 doses per day on an as-needed basis. In some embodiments, postoperative pain treatment includes a protocol for dosage of single or multiple doses over about 1 to about 5 days postoperatively, with optional further use up to about 3 weeks if pain persists.

In some embodiments, postoperative pain treatment includes a protocol for dosage of single or multiple doses over about 1 to about 5 days postoperatively, with optional further use up to about 3 weeks if pain persists. In some embodiments, procedural sedation for short term procedures includes a protocol for dosage of one administration prior to the procedure, with one or more additional administrations if the procedure is a prolonged procedure. In some embodiments, perioperative pain treatment includes a protocol for dosage single or multiple intranasal administrations before, during, or after surgery, with additional dosage sessions for up to 7 days or up to 10 weeks as needed, postoperatively.

In some embodiments, treatment of opioid withdrawal symptoms in patients undergoing buprenorphine initiation/ketamine-assisted buprenorphine Initiation includes a protocol for dosing prior to and/or during buprenorphine initiation at about 1 to about 4 doses per day for the induction period, then continued as maintenance alongside buprenorphine as needed.

In some embodiments, treatment of ketamine as adjuvant therapy for specific cancers to inhibit tumor growth and progression includes a protocol for dosage aligning with chemotherapy cycles (once per cycle) or about 1 to about 4 times monthly, where the number of administrations and duration of the period of treatment is adjusted based on tumor response and tolerability.

In some embodiments, treatment of a neurological disorder or condition such as depressive symptoms and cognitive impairment associated with Alzheimer's disease, migraine in adults, migraine in adults who are diagnosed with major depressive disorder, include a treatment protocol of dosing about 1 to about 4 times per week for about 2 to about 12 weeks, optionally followed by a maintenance period including dosing every 1 to about 8 weeks as needed. In other embodiments, treatment of certain neurological disorder or condition such as dyskinesia in Parkinson's Disease, post-traumatic insomnia, sleep disturbances in psychiatric disorders, refractory chronic migraine in patients unresponsive to standard preventive and acute migraine therapies, benzodiazepine refractory convulsive status epilepticus, and super-refractory status epilepticus include a treatment protocol of dosing about 1 to about 4 times per week for about 2 to about 12 weeks, optionally followed by a maintenance period including dosing every 1 to about 8 weeks as needed, at about 1 to about 6 doses per day on an as-needed basis.

In some embodiments, intranasally administering an effective mount of a hydro-alcoholic composition as described herein wherein the effective amount of the ketamine or pharmaceutically acceptable salt thereof is about 20 mg, and provides one or more pharmacokinetic parameters selected from the group consisting of a Cmax from about 48 to about 280 nanogram per milliliter, a Tmax from about 0.083 to about 0.5 hours, an AUC (0-1 hr) about 28.7 to about 127 h*ng/mL, an AUC (0-4 hr) from about 60.0 to about 209 h*ng/mL, and a combination thereof. In some embodiments, the effective amount of the ketamine or pharmaceutically acceptable salt thereof is 40 milligrams, and provides one or more pharmacokinetic parameter selected from the group consisting of a Cmax from about 94.7 to about 367 nanogram per milliliter, a Tmax from about 0.167 to about 0.5 hours, an AUC (0-1 hr) about 71.4 to about 225 h*ng/mL, an AUC (0-4 hr) from about 160 to about 433 h*ng/mL, and a combination thereof. These parameters may be achieved by the hydro-alcoholic composition described herein, for example, a hydro-alcoholic composition comprising ketamine or a pharmaceutically acceptable salt thereof in an amount of about 17 wt % to about 20 wt %; n-dodecyl-β-D-maltoside in an amount of about 0.3 wt % to about 0.8 wt %; propylene glycol in an amount of about 3 wt % to about 7 wt %; ethyl alcohol in an amount of about 3 wt % to about 7 wt %; benzalkonium chloride in an amount of about 0.01 wt % to about 0.05 wt %; ethylenediamine tetraacetate in an amount of about 0.01% to about 0.05%; and water, wherein the composition has a pH of about 3 to about 5, or the hydro-alcoholic composition as described in Table 1.

In some embodiments, intranasally administering an effective mount of a hydro-alcoholic composition as described herein, wherein the effective amount of esketamine or a pharmaceutically acceptable salt is about 10 mg, and provides one or more pharmacokinetic parameters selected from the group consisting of a Cmax from about 21.4 to about 125 nanogram per milliliter, a Tmax from about 0.0833 to about 0.33 hours, an AUC (0-1 hr) about 12.8 to about 55.8 h*ng/mL, an AUC (0-4 hr) from about 26.4 to about 90.4 h*ng/mL, and a combination thereof. In some embodiments, the effective amount of the esketamine or pharmaceutically acceptable salt thereof is 20 milligrams of esketamine or a salt thereof, and provides one or more pharmacokinetic parameters selected from the group consisting of a Cmax from about 41.3 to about 161 nanogram per milliliter, a Tmax from about 0.167 to about 0.5 hours, an AUC (0-1 hr) about 30.9 to about 96.7 h*ng/mL, an AUC (0-4 hr) from about 66.9 to about 181 h*ng/mL, and a combination thereof. These parameters may be achieved by the hydro-alcoholic composition described herein, for example, a hydro-alcoholic composition comprising ketamine or a pharmaceutically acceptable salt thereof in an amount of about 17 wt % to about 20 wt %; n-dodecyl-β-D-maltoside in an amount of about 0.3 wt % to about 0.8 wt %; propylene glycol in an amount of about 3 wt % to about 7 wt %; ethyl alcohol in an amount of about 3 wt % to about 7 wt %; benzalkonium chloride in an amount of about 0.01 wt % to about 0.05 wt %; ethylenediamine tetraacetate in an amount of about 0.01% to about 0.05%; and water, wherein the composition has a pH of about 3 to about 5, or the hydro-alcoholic composition as described in Table 1.

In some embodiments, the bioavailability of the ketamine or esketamine may be determined by analyzing the blood plasma of a subject administered a hydro-alcoholic composition as described herein. In some embodiments, the bioavailability of the ketamine or esketamine after intranasal administration may be compared to intramuscular and/or intravenous administration of approved products.

Examples of the pharmaceutically acceptable carriers include an antioxidant, a stabilizer, a preservative, a taste-masking agent, a colorant, a solubilizer, a solubilizing agent, a surfactant, an emulsifier, an anti-foaming agent, a viscosity adjustor, a gelling agent, an absorption accelerator, a dispersant, an excipient, and a pH adjustor.

### Statement of Certain Embodiments of the Invention

1. In a first embodiment, a hydro-alcoholic composition comprises ketamine or a pharmaceutically acceptable salt thereof, wherein the composition comprises about 5 wt % to about 25 wt % ketamine or a pharmaceutically acceptable salt thereof; a permeation enhancing agent comprising an alkyl glycoside, wherein the composition comprises about 0.1 wt % to about 20 wt % alkyl glycoside; about 1 wt % to about 20 wt % alky glycol; about 1 wt % to about 20 wt % ethanol; optionally, a preservative, a stabilizing agent, and/or a buffering agent; and a pharmaceutically acceptable excipient comprising water, wherein the composition comprises about 40 wt % to about 90 wt % water and the hydro-alcoholic composition has a pH of about 3 to about 6.5.

2. The hydro-alcoholic composition of embodiment 1, wherein the composition comprises about 15 wt % to about 22 wt % ketamine or a pharmaceutically acceptable salt thereof.

3. The hydro-alcoholic composition of embodiment 1 or 2, wherein the composition comprises about 17 wt % to about 19 wt % ketamine or a pharmaceutically acceptable salt thereof.

4. The hydro-alcoholic composition of any one of embodiments 1-3, wherein the composition comprises about 17 wt % to about 18 wt % ketamine or a pharmaceutically acceptable salt thereof.

5. The hydro-alcoholic composition of any one of embodiments 1-4, wherein the alkyl glycoside is selected from the group consisting of dodecyl maltoside (n-dodecyl-β-D-maltoside), 6-Cyclohexyl-1-hexyl-β-D-maltopyranoside, decyl maltoside (n-decyl-β-D-maltopyranoside), octyl glucoside (n-octyl-β-d-glucoside), decyl glucoside (decyl β-D-glucopyranoside), and lauryl glucoside (dodecyl β-D-glucopyranoside).

6. The hydro-alcoholic composition of any one of embodiments 1-5, wherein the composition comprises about 0.1 wt % to about 1.5 wt % alkyl glycoside.

7. The hydro-alcoholic composition of any one of embodiments 1-6, wherein the composition comprises about 0.1 wt % to about 1 wt % alkyl glycoside.

8. The hydro-alcoholic composition of any one of embodiments 1-7, wherein the composition comprises about 0.1 wt % to about 0.8 wt % alkyl glycoside.

9. The hydro-alcoholic composition of any one of embodiments 1-8, wherein the composition comprises about 0.25 wt % to about 1 wt % alkyl glycoside.

10. The hydro-alcoholic composition of any one of embodiments 1-9, wherein the composition comprises about 0.5 wt % to about 0.75 wt % alkyl glycoside.

11. The hydro-alcoholic composition of any one of embodiments 1-10, wherein the composition comprises about 0.5 wt % or about 0.75 wt % alkyl glycoside.

12. The hydro-alcoholic composition of any one of embodiments 1-11, wherein the composition comprises about 0.5 wt % alkyl glycoside.

13. The hydro-alcoholic composition of any one of embodiments 1-12, wherein the alkyl glycoside is n-dodecyl-β-D-maltoside.

14. The hydro-alcoholic composition of any one of embodiments 1-13, wherein the alkyl glycol is selected from the group consisting of ethylene glycol, propylene glycol, butylene glycol, and pentylene glycol.

15. The hydro-alcoholic composition of any one of embodiments 1-14, wherein composition comprises about 1 wt % to about 10 wt % alkyl glycol, and the alkyl glycol comprises propylene glycol.

16. The hydro-alcoholic composition of any one of embodiments 1-15, wherein the composition comprises about 3 wt % to about 7 wt % alkyl glycol, and the alkyl glycol comprises propylene glycol.

17. The hydro-alcoholic composition of any one of embodiments 1-16, wherein the composition comprises about 5 wt % alkyl glycol, and the alkyl glycol comprises propylene glycol.

18. The hydro-alcoholic composition of any one of embodiments 1-17, wherein the composition comprises about 1 wt % to about 10 wt % ethanol.

19. The hydro-alcoholic composition of any one of embodiments 1-18, wherein the composition comprises about 3 wt % to about 7 wt % ethanol.

20. The hydro-alcoholic composition of any one of embodiments 1-19, wherein the composition comprises about 5 wt % ethanol.

21. The hydro-alcoholic composition of any one of embodiments 1-20, wherein the preservative agent is present in an amount of about 0.01 wt % to about 0.5 wt %.

22. The hydro-alcoholic composition of any one of embodiments 1-21, wherein the preservative agent is present in an amount of about 0.01 wt % to about 0.1 wt %.

23. The hydro-alcoholic composition of any one of embodiments 1-22, wherein the preservative agent comprises benzalkonium chloride; and the stabilizing agent comprises ethylenediamine tetraacetate (EDTA).

24. The hydro-alcoholic composition of any one of embodiments 1-23, wherein the benzalkonium chloride is present in an amount of about 0.01 wt % to about 0.1 wt %.

25. The hydro-alcoholic composition of any one of embodiments 1-24, wherein, the EDTA is present in the composition in an amount of about 0.01 wt % to about 0.1 wt %.

26. The hydro-alcoholic composition of any one of embodiments 1-25, wherein the benzalkonium chloride is present in the composition in an amount of about 0.02 wt % and the EDTA is present in the composition in an amount of about 0.05 wt %.

27. The hydro-alcoholic composition of any one of embodiments 1-26, wherein the pharmaceutically acceptable excipient is water, and the composition comprises about 66 wt % to about 78 wt % water.

28. The hydro-alcoholic composition of any one of embodiments 1-27 consisting essentially of: about 15 wt % to about 20 wt % ketamine or a pharmaceutically acceptable salt thereof; about 0.3 wt % to about 1 wt % n-dodecyl-β-D-maltoside; about 3 wt % to about 7 wt % propylene glycol; about 3 wt % to about 7 wt % ethyl alcohol; about 0.01 wt % to about 0.05 wt % benzalkonium chloride; about 0.01% to about 0.1% ethylenediamine tetraacetate; optionally, one or more buffering agents; and water, wherein the composition has a pH of about 3 to about 6.

29. The hydro-alcoholic composition of any one of embodiments 1-28 consisting essentially of: about 17 wt % to about 19 wt % ketamine or a pharmaceutically acceptable salt thereof; about 0.5 wt % to about 0.75 wt % n-dodecyl-β-D-maltoside; about 5 wt % propylene glycol; about 5 wt % ethyl alcohol; about 0.02 wt % benzalkonium chloride; about 0.05% ethylenediamine tetraacetate; optionally, one or more buffering agents; and water, wherein the composition has a pH of about 3 to about 6.

30. A hydro-alcoholic composition consisting of: about 15 wt % to about 20 wt % ketamine or a pharmaceutically acceptable salt thereof, about 0.1 wt % to about 1.5 wt % n-dodecyl-β-D-maltoside; about 1 wt % to about 10 wt % propylene glycol; about 1 wt % to about 10 wt % ethyl alcohol; about 0.01 wt % to about 0.05 wt % benzalkonium chloride; about 0.01% to about 0.1% ethylenediamine tetraacetate; optionally, one more buffering agents; and water, wherein the composition has a pH of about 3 to about 6.

31. The hydro-alcoholic composition of any one of embodiments 1-30, wherein the ketamine is the S-enantiomer of ketamine (esketamine) or a pharmaceutically acceptable salt thereof.

32. The hydro-alcoholic composition of any one of embodiments 1-30, wherein the ketamine is the R-enantiomer of ketamine (arketamine) or a pharmaceutically acceptable salt thereof.

33. The hydro-alcoholic composition of any one of embodiments 1-32, wherein the composition is formulated as a nasal spray formulation.

The following Examples are intended to illustrate the above inventions and should not be construed as to narrow its scope. One skilled in the art will readily recognize that the Examples suggest many other ways in which the inventions could be practiced. It should be understood that numerous variations and modifications may be made while remaining within the scope of the inventions.

### EXAMPLES

### Example 1. Ketamine Nasal Spray Formula, 20 mg/Spray

In one embodiment, the Es/ketamine Nasal Spray is a non-aqueous (hydro-alcoholic) formulation containing 15-20% w/w Es/ketamine HCl wherein the optimum water level is 66.83-77.58% w/w. Ethanol and Intravail® (n-dodecyl β-D-maltoside) are included in the formulation to enhance membrane permeability and bioavailability. Propylene Glycol is included to reduce possible nasal irritation and to further improve membrane absorption. BAK and EDTA are used to improve stability. A buffer may be used to maintain the optimum pH for drug absorption and drug stability. The components of one specific embodiment of the Es/ketamine Nasal Spray are shown in Table 1 below.

TABLE 1
An es/ketamine nasal spray formulation,
according to one specific embodiment.

| Ingredient | Function | % w/w |
|---|---|---|
| Es/ketamine Hydrochloride, | Active Pharmaceutical | 17.1 |
| USP | Ingredient | |
| Intravail ® (n-Dodecyl β- | Permeation Enhancer | 0.5 |
| D-Maltoside) | (improves bioavailability) | |
| Dehydrated Ethyl Alcohol, | Permeation Enhancer/Cosolvent | 5 |
| USP | (improves onset of action and | |
| | bioavailability) | |
| Propylene Glycol, USP | Cosolvent | 5 |
| Benzalkonium Chloride, | Preservative (aids in product | 0.02 |
| 50% USP | stability) | |
| EDTA, USP | Chelating agent (aids in product | 0.05 |
| | stability) | |
| 1N NaOH | pH adjustment (maintains pH | Adjust to |
| | between 4-5; aids in stability) | pH 4.5 |
| Purified Water, USP | Vehicle | QS to 100 |

Es/ketamine HCl (API) concentration was maximized to increase the amount of drug delivered per spray. At 17.1% w/w Es/ketamine HCl, each 130 μL spray (multidose device) delivers 20 mg of Es/ketamine Base. For the 100 μL unidose device, delivery would be 15 mg Es/ketamine Base per spray. Concentrations from approximately 14.6 w/w to 20% w/w Es/ketamine HCl were evaluated. This full range from 14.6% to 20% is acceptable for use depending on co-solvents used and the amount of drug needed per spray.

Intravail® brand permeation enhancer and Dehydrated Ethyl Alcohol are included in the formulation to improve membrane permeation and to assist with solubility. One of the primary keys to increasing bioavailability is increasing membrane permeation. Membrane permeation studies (Franz Cell) were conducted with varying levels of ethyl alcohol (0 to 10% w/w), Intravail®, (0 to 0.75% w/w), and water (66.83-77.58% w/w). These studies show that, independently, alcohol and Intravail® improve membrane permeation. However, the improvement was enhanced even further (+50%) when used in combination. It was determined that a concentration of 5% ethanol and 0.5% Intravail® provided the best level of membrane permeation. However, Intravail® levels between 0.25% to 0.75% do enhance permeation to acceptable levels and any level of Ethanol up to 10% is beneficial. At levels above 10% ethanol, the solubility of excipients may become problematic.

Higher levels of Intravail® or higher levels of ethanol did not improve permeation significantly. Also, the removal of ethanol or Intravail® resulted in less than optimum permeation. The formulation without ethanol and formulation without Intravail® exhibited very similar release kinetics and exhibited lower drug permeation. On the other hand, formulation with ethanol alone exhibited poor drug permeation. However, formulations containing both Intravail® and ethanol exhibited better drug permeation. Among the formulations containing both Intravail® and ethanol, formulation containing 5% Ethanol and 0.5% Intravail® provided the best overall permeation though porcine nasal transmucosal membrane. The possible reason might be the formation of drug-micellar complexes at a concentration of 0.5% Intravail® and in presence of water:ethanol co-solvent system.

Generally, increase in the concentration of permeation enhancers increase the absorption up to certain concentration levels but beyond the optimal concentration it retards the absorption. Steric effects exerted by high concentrations might hinder drug transport through the membrane. We have observed this with n-Dodecyl β-D-Maltoside with Ketamine. In a previously published study, n-Dodecyl β-D-Maltoside exhibited similar transport inhibition of Octreotide at higher concentrations (see for example, Maher et al., Advanced Drug Delivery Reviews, 177 (2021) 113925). Increase in the ethanol concentration in the formulation, generally increase the permeation, however we observed decreased permeation when ethanol concentrations increased from 5% to 10% and this result might be due to decreased solubility of ketamine and Intravail® in solvent system when the ethanol concentration increases.

Propylene Glycol is commonly used in nasal sprays as a nasal lubricant. It reduces irritation in the nasal passages. Propylene Glycol helps add moisture inside the nose which helps keep the nasal membrane clear of mucus and aids in absorption of the medication. In addition, propylene glycol helps to counteract the drying effects of ethanol.

Benzalkonium Chloride is used as a preservative. Benzalkonium chloride (BKC) is a quaternary ammonium compound commonly used to prevent bacterial contamination and to preserve pharmacological activity in nasal sprays. In both human and veterinary nasal formulations, one of the most common and effective preservatives is Benzalkonium Chloride (BKC/BAK). Benzalkonium Chloride is widely used as an excipient in nasal sprays, nasal drops, and ointments, where its concentration typically varies from 0.005% up to 0.2%. Antimicrobial effectiveness of the formulated level of BAK (0.02%) was conducted and found acceptable. The preservative is optional.

EDTA (ethylene-diamine-tetra-acetic acid) is commonly used as a preservative in nasal sprays, as well as in other drug products and medical devices. It acts as a chelating agent, which means it can bind to metal ions and prevent their degradation or interaction with other compounds. In nasal sprays, EDTA helps to enhance the stability of the formulation by chelating metal ions that may catalyze degradation reactions. This helps to maintain the effectiveness and shelf-life of the product. The preservative is optional.

A Buffer may be used to control the pH of the nasal spray solution at, or near 4.5. Es/ketamine HCl solutions have shown better stability at an acidic pH near 4.5. Also, the local pH value inside the nasal cavity directly influences the rate and extent of drug absorption. It is suggested that the optimum pH value of a nasal spray ranges from 4.5 to 6.5. Additionally, maintaining pH at or above 4.0 helps to reduce local nasal tract irritation. The buffer is optional.

### Example 2. Absolute Bioavailability of Ketamine Intranasal Formulation

Ketamine [(±)-2-(o-Chlorophenyl)-2-methylamino) cyclohexanone hydrochloride] is a nonbarbiturate general anesthetic. Ketamine hydrochloride is a white crystalline powder and has a molecular formula of C₁₃H₁₆ClNO·HCl and a molecular weight of 274.19. Ketamine is approved for intravenous or intramuscular injection as an anesthetic agent for diagnostic and surgical procedures for the induction of anesthesia prior to the administration of other general agents and to supplement other anesthetic agents.

In some situations, IN drug delivery is a preferable alternative to intramuscular or intravenous administration as it is a practical, noninvasive, and convenient dosing option. It provides rapid onset (potentially equivalent to IM or IV), is not subject to first pass metabolism, and should have good bioavailability and fewer application site-related side effects.

This study evaluated a new intranasal (IN) formulation of ketamine to determine the dose that should provide comparable exposure to intravenous and/or intramuscular injection.

### Study Design.

This was an open-label study in 25 healthy volunteers. Subjects received single doses of ketamine by intravenous (IV) injection (20 mg), intramuscular (IM) injection (20 mg), or intranasal (IN) administration (20 mg, 40 mg and 80 mg).

Blood samples for the measurement of plasma concentrations of (R)- and (S)-ketamine and (R)- and (S)-norketamine were collected pre-dose and at 0.033, 0.083, 0.167, 0.25, 0.333, 0.5, 0.75, 1, 1.25, 2, 2.5, 3, 4, 6, 8, 12, 16, and 24 hours post-dose for each study period.

### Overall Summary of Absolute Bioavailability

The absolute bioavailability of ketamine after IN administration relative to IV administration is summarized in the table below. All estimates for the parent and metabolite were consistent within each formulation or dose for the racemate, (R)-, and (S)-enantiomers. After IN administration, the estimates were slightly higher for the metabolite than the parent.

TABLE 3
Summary of Absolute Bioavailability Estimates
for IN Administration of Racemic Ketamine

| | Absolute Bioavailability (%)* | | |
|---|---|---|---|
| Analyte | 20 mg | 40 mg | 80 mg |
| (R)-Ketamine | 81.6 | 68.6 | 51.1 |
| (S)-Ketamine | 82.4 | 68.9 | 51.5 |
| (RS)-Ketamine | 82.1 | 69.1 | 51.8 |
| (R)-Norketamine | 90.7 | 77.8 | 54.3 |
| (S)-Norketamine | 92.4 | 77.8 | 54.9 |
| (RS)-Norketamine | 94.4 | 78.8 | 54.8 |

*Based on the dose-corrected ratio of the least squares geometric mean AUC_{(inf)} from the statistical analyses.

TABLE 4
Pharmacokinetic parameters of the hydro-alcoholic composition.

| | | | | |
|---|---|---|---|---|
| Dose | Tmax | Cmax | AUC_{(0-1 hr)} | AUC_{(0-4 hr)} |
| (mg) | (hr) | (ng/mL) | (hr′ng/mL) | (hr′ng/mL) |

(RS)-Ketamine

| | | | | |
|---|---|---|---|---|
| 20 | 0.083-0.50 | 48.0-280 | 28.7-127 | 60.0-209 |
| 40 | 0.167-0.50 | 94.7-367 | 71.4-225 | 160-433 |

(S)-Ketamine

| | | | | |
|---|---|---|---|---|
| 10 | 0.0833-0.333 | 21.4-125 | 12.8-55.8 | 26.4-90.4 |
| 20 | 0.167-0.50 | 41.3-161 | 30.9-96.7 | 66.9-181 |

(R)-Ketamine

| | | | | |
|---|---|---|---|---|
| 10 | 0.083-0.50 | 26.5-155 | 15.9-70.8 | 33.6-118 |
| 20 | 0.167-0.50 | 53.4-206 | 40.5-129 | 93.0-251 |

Notes:
Values are presented as the minimum-maximum.

In some embodiments, up to 20 mg/spray may be administered vs. other esketamine nasal sprays which deliver approximately 14 mg/spray.

### Example 3. Permeation Characteristics of the Formulations

The formulations described herein provide significantly improved ketamine membrane permeation and nasal bioavailability. These results are surprising at least in part because the prevailing art would suggest a person of ordinary skill in the art would expect continued increases in Intravail concentrations and/or alcohol concentrations would further improve permeation. However, the inventors found that a synergistic ratio of Intravail and Ethanol provides the best overall permeation.

Typically, one would expect membrane permeation to increase with increased levels of Intravail (perhaps up to 0.75%) and to also increase with increased levels of ethanol up to possibly 20%. The inventors' formulations unexpectedly display characteristics that contradict the prevailing art. As described in various permeation studies, membrane permeation increased with Intravail concentrations from 0.25 to 0.75%. Membrane permeation increased with increased ethanol concentrations up to 5%, but a higher concentration of ethanol (10%) significantly reduced permeation. Intravail+Ethanol in combination yielded membrane permeation significantly above what was observed with Intravail alone (up to 0.75%) and ethanol alone (up to 10%).

It is this synergy between Intravail and Ethanol that is unique. For this specific formulation, it appears that high levels of ethanol negatively impact solubility, causing a reduction in membrane permeation. While 0.75% Intravail with 5% Ethanol produces the second highest level of membrane permeation, a reduction to 0.5% Intravail increases permeation even further (±10%). Intravail is a non-ionic surfactant with a hydrophilic maltose head and a hydrophobic long chain alkyl tail and helps the drug molecule pass through the membrane by altering the membrane bound proteins. Ethanol is also known to disrupt the membrane proteins and help in drug absorption. High permeability was observed at 0.5% Intravail in a hydroalcoholic system containing 5% ethanol. The possible reason for high drug absorption at this synergistic ratio might be the formation of drug-micellar complexes which aid in higher drug permeation through the transmucosal membrane.

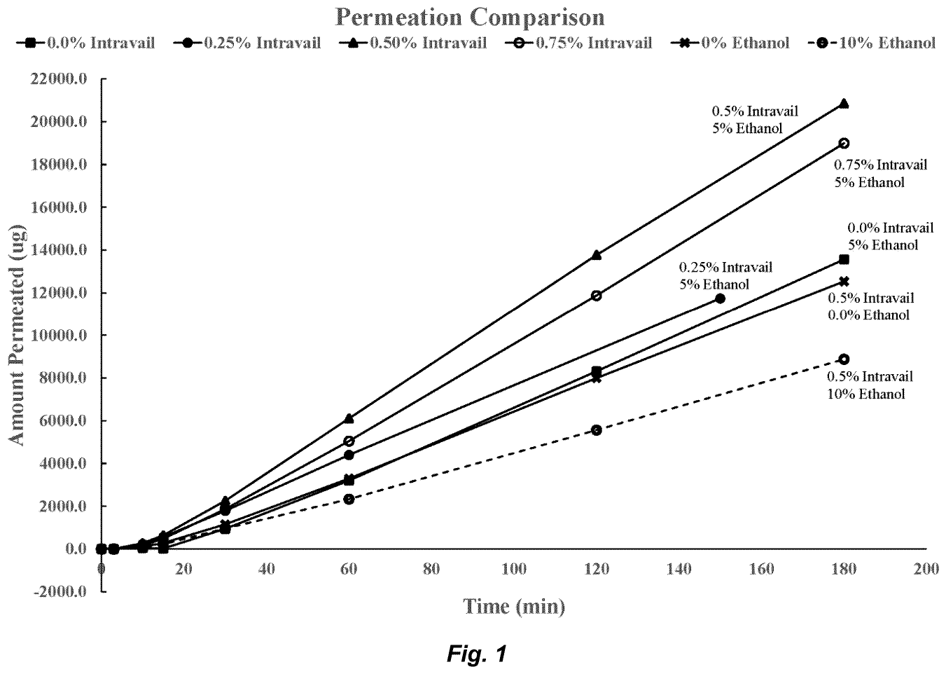

It was expected that higher levels of Intravail and higher levels of ethanol would increase membrane permeation. However, as shown in the permeation study (FIG. 1), the ketamine formulation containing 5% Ethanol and 0.5% Intravail provided the best overall permeation though the porcine nasal membrane. Higher levels of Intravail or higher levels of ethanol did not improve permeation. The reduction or removal of ethanol or Intravail resulted in less than optimum permeation (See FIG. 1).

All publications, patents, and patent documents cited herein are incorporated by reference as though individually incorporated by reference, and in particular EP Patent Publication EP 4154866 to Savmarker et al.; U.S. Patent Publication No. 2023/0121313 to Jimidar et al.; U.S. Patent Publication No. 2023/0000796 to Holm et al.; U.S. Patent Publication No. 2024/0000728 to Drevets et al.; U.S. Patent Publication No. 2020/0129402 to Narahari et al.; U.S. Pat. No. 11,241,414 to Cartt et al.; U.S. Pat. No. 10,265,402 to Maggio et al.; U.S. Pat. No. 11,446,260 to Bastannie et al.; and U.S. Pat. No. 11,771,661 to Charney et al. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A hydro-alcoholic composition comprising:
ketamine or a pharmaceutically acceptable salt thereof, wherein the composition comprises about 5 wt % to about 25 wt % ketamine or a pharmaceutically acceptable salt thereof;
a permeation enhancing agent comprising an alkyl glycoside, wherein the composition comprises about 0.1 wt % to about 20 wt % alkyl glycoside;
about 1 wt % to about 20 wt % alky glycol;
about 1 wt % to about 20 wt % ethanol;
optionally, a preservative agent, a stabilizing agent, and/or a buffering agent; and
a pharmaceutically acceptable excipient comprising water, wherein the composition comprises about 40 wt % to about 90 wt % water;
wherein the hydro-alcoholic composition has a pH of about 3 to about 6.5.

2. The hydro-alcoholic composition of claim 1, wherein the composition comprises about 15 wt % to about 22 wt % ketamine or a pharmaceutically acceptable salt thereof.

3. The hydro-alcoholic composition of claim 2, wherein the composition comprises about 17 wt % to about 19 wt % ketamine or a pharmaceutically acceptable salt thereof.

4. The hydro-alcoholic composition of claim 1, wherein the alkyl glycoside is selected from the group consisting of dodecyl maltoside (n-dodecyl-β-D-maltoside), 6-cyclohexyl-1-hexyl-β-D-maltopyranoside, decyl maltoside (n-decyl-β-D-maltopyranoside), octyl glucoside (n-octyl-β-d-glucoside), decyl glucoside (decyl β-D-glucopyranoside), and lauryl glucoside (dodecyl 3-D-glucopyranoside).

5. The hydro-alcoholic composition of claim 1, wherein the composition comprises about 0.1 wt % to about 1.5 wt % alkyl glycoside.

6. The hydro-alcoholic composition of claim 5, wherein the composition comprises about 0.1 wt % to about 1 wt % alkyl glycoside.

7. The hydro-alcoholic composition of claim 1, wherein the alkyl glycoside is n-dodecyl-β-D-maltoside.

8. The hydro-alcoholic composition of claim 1, wherein the alkyl glycol is selected from the group consisting of ethylene glycol, propylene glycol, butylene glycol, and pentylene glycol.

9. The hydro-alcoholic composition of claim 1, wherein composition comprises about 1 wt % to about 10 wt % alkyl glycol, and the alkyl glycol comprises propylene glycol.

10. The hydro-alcoholic composition of claim 9, wherein the composition comprises about 3 wt % to about 7 wt % alkyl glycol, and the alkyl glycol comprises propylene glycol.

11. The hydro-alcoholic composition of claim 10, wherein the composition comprises about 5 wt % alkyl glycol, and the alkyl glycol comprises propylene glycol.

12. The hydro-alcoholic composition of claim 1, wherein the composition comprises about 1 wt % to about 10 wt % ethanol.

13. The hydro-alcoholic composition of claim 12, wherein the composition comprises about 3 wt % to about 7 wt % ethanol.

14. The hydro-alcoholic composition of claim 1, wherein ketamine is the S-enantiomer of ketamine (esketamine) or a pharmaceutically acceptable salt thereof.

15. The hydro-alcoholic composition of claim 1, wherein the ketamine is the R-enantiomer of ketamine (arketamine) or a pharmaceutically acceptable salt thereof.

16. The hydro-alcoholic composition of claim 1, wherein the preservative agent comprises benzalkonium chloride; and the stabilizing agent comprises ethylenediamine tetraacetate (EDTA).

17. The hydro-alcoholic composition of claim 15, wherein the benzalkonium chloride is present in the composition in an amount of about 0.01 wt % to about 0.05 wt %; and the EDTA is present in the composition in an amount of about 0.01 wt % to about 0.1 wt %.

18. The hydro-alcoholic composition of claim 1, wherein the pharmaceutically acceptable excipient is water and the composition comprises about 66 wt % to about 78 wt % water.

19. The hydro-alcoholic composition of claim 1 consisting essentially of:
about 15 wt % to about 20 wt % ketamine or a pharmaceutically acceptable salt thereof,
about 0.3 wt % to about 1 wt % n-dodecyl-β-D-maltoside;
about 3 wt % to about 7 wt % propylene glycol;
about 3 wt % to about 7 wt % ethyl alcohol;
about 0.01 wt % to about 0.05 wt % benzalkonium chloride;
about 0.01% to about 0.1% ethylenediamine tetraacetate;
optionally, one or more buffering agents; and
water, wherein the composition has a pH of about 3 to about 6.

20. The hydro-alcoholic composition of claim 1 consisting essentially of:
about 17 wt % to about 19 wt % ketamine or a pharmaceutically acceptable salt thereof;
about 0.5 wt % to about 0.75 wt % n-dodecyl-β-D-maltoside;
about 5 wt % propylene glycol;
about 5 wt % ethyl alcohol;
about 0.02 wt % benzalkonium chloride;
about 0.05% ethylenediamine tetraacetate;
optionally, one or more buffering agents; and
water, wherein the composition has a pH of about 3 to about 6.

21. A hydro-alcoholic composition consisting of:
about 15 wt % to about 20 wt % ketamine or a pharmaceutically acceptable salt thereof,
about 0.1 wt % to about 1.5 wt % n-dodecyl-β-D-maltoside;
about 1 wt % to about 10 wt % propylene glycol;
about 1 wt % to about 10 wt % ethyl alcohol;
about 0.01 wt % to about 0.05 wt % benzalkonium chloride;
about 0.01% to about 0.1% ethylenediamine tetraacetate;
optionally, one more buffering agents; and
water, wherein the composition has a pH of about 3 to about 6.
